SEQUENCE LISTING (1 sequence)

SEQ ID NO: 1 - DNA, 771 bp, Bombyx mori
atgagactgactttgtttgccttcgtcctcgccgtgtgtgcgctggcttctaacgccacacttgcaccaagaactgatgacgtactggcagagcagctgtatatgagtgtcgtcattggtgaatacgagaccgctatcgccaaatgctctgaatatctgaaggaaaagaagggagaggttatcaaggaagccgtgaagcgtctgatcgaaaacggcaagaggaacaccatggacttcgcctaccagttatggacaaaggatggaaaggaaatcgtcaaatcttacttccccatccagtttagagtgatcttcaccgagcagactgtcaagctcataaacaaaagggaccatcacgccctcaagttgatcgaccaacaaaaccacaacaaaattgcattcggtgactccaaagacaaaaccagcaagaaagtctcctggaagtttacccccgtgttggaaaacaacagagtatacttcaagatcatgtccaccgaagacaaacagtacctgaagctcgataacacgaaaggttctagtgatgaccgtatcatctacggtgatagcaccgctgacaccttcaaacaccactggtaccttgagccctccatgtacgaaagcgacgtcatgttcttcgtctacaaccgagagtacaacagtgttatgacacttgatgaagatatggccgccaacgaagaccgtgaagccttggggcacagcggagaagtttccggttatccccaactttttgcatggtacatcgtcccctactaa

(19) United States
(12) Patent Application Publication
(10) Pub. No.: US 2006/0078965 A1
(43) Pub. Date: Apr. 13, 2006

(54) Chinese hamster ovary cell line transfected with 30K gene

(76) Inventors: Tai Hyun Park, Seoul (KR); Shin Sik Choi, Seoul (KR)

Correspondence Address: NATH & ASSOCIATES, 112 South West Street, Alexandria, VA, 22314, US

(21) Appl. No.: 10/963,770

(22) Filed: Oct. 14, 2004

(30) Foreign Application Priority Data
Oct. 10, 2004 (KR) 10-2004-0080728

(51) Int. Cl. C12N 5/06; C12P 21/06

(52) U.S. Cl. 435/69.1; 435/358; 435/320.1

(57) ABSTRACT

The present invention relates to a CHO (Chinese hamster ovary) cell line transfected with 30 K gene. More particularly, the present invention is directed to a CHO cell line transfected with 30 K genes obtained from the silkworm Bombyx Mori, which has anti-apoptotic property.

### FIELD OF INVENTION

The present invention relates to a CHO (Chinese hamster ovary) cell line transfected with 30 K gene. More particularly, the present invention is directed to a CHO cell line transfected with 30 K genes obtained from the silkworm Bombyx Mori, which has anti-apoptotic property.

### DESCRIPTION OF THE RELATED ART

The CHO cell line of the present invention means a cell line obtained from Chinese hamster ovary, has been verified safety and stability, and thus can be easily approved by supervisory institutions such as the FDA in USA.

In the field of biology and medical science, a desired target protein can be obtained mainly by culturing transfected cell lines. The methods using CHO (Chinese Hamster Ovary) cell line, BHK (Baby Hamster Kidney) cell line, and NSO cell line (Murine myeloma cell line) are examples used for the production of target proteins in the industry (Ogata, et al., Applied Microbiology and Biotechnology, 1993, 38(4), 520-525; Kratje, et al., Biotechnology Progress, 1994, 10(4), 410-20; Peakman, et al., Human Antibodies Hybridomas, 1994, 5(1-2), 65-74).

Among the above cell lines, the CHO cell line is the most effectively used host cell line in the industry for the mass-production of target proteins using animal cells. There are five main reasons that the CHO cell line is industrially preferred:
(i) The posttranslational modification process of protein, that is, glycosylation or phosphorylation process, is similar to that of the human cells;
(ii) Suspension culturing as well as adhesion culturing of the cell is possible;
(iii) Relatively high concentrations of cells can be achieved compared with other cell lines cultured in a serum-free culture medium;
(iv) The productivity of the target protein, which is significantly lower than that of other microorganisms, can be increased by the dihydrofolate reductase/methotrexate (DHFR/MTX) amplifying system; and
(v) Since safety and stability of the CHO cell line has been verified, the cell line can be easily approved by supervisory institutions such as the FDA.

Recombinant CHO cell lines can be prepared by transfecting a target gene into the CHO cell line. To mass-produce target proteins industrially using a recombinant CHO cell line, the recombinant CHO cells should be cultured as suspended forms in culture medium.

By the way, the serum employed in the culture medium, may contain various unidentified proteins which should not be allowed for the preparation of pharmaceutical formalation. Therefore, in order to employ the serum as a component of the cell culture medium, the serum should be treated through expensive refining process.

In addition, recently, health supervisory institutions such as the FDA require the exclusion of serum throughout the entire process due to an outbreak of mad cow disease. When the CHO cell line is cultured as a suspended form in a serum-free culture media; however, the amount of produced target protein tends to decrease due to the apoptosis (Itoh, et al., Biotechnology and Bioengineering, 1995, 48, 118-122; Suzuki, et al., Cytotechnology, 1997, 23, 55-59; Simpson, et al., Biotechnology and Bioengineering, 1997, 54, 1-16).

Furthermore, the decrease in survival rate caused by programmed cell death not only lowers the productivity of target proteins but also affects the stability of target proteins as various proteases, present inside the cells, get secreted by cell lysis. Thus, the DNA and cell debris of the lysed cells complicate the subsequent purifying process.

In addition, when sodium butyrate (NaBu) is added in order to increase the amount of target proteins, apoptosis tends to be increased.

The mechanism of programmed cell death is as follows. When the initiator caspase, a kind of protease, is activated by various stimuli, the membrane potential of mitochondria is disintegrated. Thereafter, cytochrome C, which is involved in the electron transfer system of mitochondria, is released from the cytoplasm. Cytochrome C released into the cytoplasm activates the effector caspase such as caspase 3, and thus, phophatidylserine, one of the main components of the phospholipid in the cell membrane, flips towards the cytoplasm. Accordingly, the DNA is digested by the activated endonuclease, and thus, the cell eventually undergoes apoptosis.

Meanwhile, an apoptosis-inhibiting component of silkworm hemolymph, was isolated and characterized in the present inventor's previous research USA patent application Ser. No. 10/926,406 and Korean Patent Application No. 10-20020059686 of the present inventors disclose the facts that the expression of 30 K inhibited apoptosis comparably to that of whole silkworm hemolymph and that both intracellular expression and external supplementation inhibited apoptosis.

### SUMMARY OF THE INVENTION

The primary purpose of the present invention is to provide a Chinese hamster ovary cell line (KCLRF-BP-00103) transfected lath 30 K gene of SEQ. ID. No. 1, which has anti-apoptotic property.

The present inventors prepared CHO cell lines transfected with 30 K genes, which is obtained from silkworm, coding for the anti-apoptotic 30 K proteins, and showed that the apoptosis can be decreased and consequently the target protein can be mass produced by employing the anti-apoptotic CHO cell line of the present invention.

Another object of the present invention is to provide the Chinese hamster ovary cell according to claim 1, which further containing a gene encoding hematopoietic growth factor.

It is a still another object of the present invention to provide the Chinese hamster ovary cell lute according to claim 2, wherein said hematopoietic growth factor is erythropoietin (EPO) protein.

### BRIEF DESCRIPTION OF THE DRAWINGS

The above objects and other advantages of the present invention will become more apparent by describing in detail a preferred embodiment thereof with reference to the attached drawings, in which:

FIG. 1 is the photograph of RT-PCR analysis of 30 K mRNA in the stable CHO cell lines transfected with 30 K expression construct.

FIG. 2 is a set of graphs comparing cell concentration of the CHO cell line overexpressing 30Kc6 protein with those of a control group of CHO cells cultured in serum-free medium.

FIG. 3a is a set of graphs comparing EPO production per medium volume in serum-free medium.

FIG. 3b is a set of graphs comparing EPO production per cells in serum-free medium.

FIG. 4 is a photograph of two-dimensional electrophoresis of EPO samples.

FIG. 5 is a photograph of lectin binding assay of EPO samples.

### DETAILED DESCRIPTION OF THE INVENTION

The object of the present invention can be achieved by providing a Chinese hamster ovary cell line (KCLRF-BP-00103) transfected with 30 K gene of SEQ. ID. No.1, which has anti-apoptotic proterty.

It has been shown that silkworm hemolymph inhibits apoptosis in insect, mammalian, and human cell systems. These results indicate that silkworm hemolymph contains a component that inhibits apoptosis. More recently, this anti-apoptotic fraction was isolated from silkworm hemolymph and characterized by the present inventors.

The fraction of silkworm hemolymph with the highest activity was found to contain 30 K proteins, which are a specific type of plasma protein called “storage proteins”. These proteins constitute a group of structurally related proteins of approximate molecular weight 30,000 Da. The 30 K protein group consists of five proteins, (30Kc6 (GenBank Accession No.:X07552), 30Kc12 (GenBank Accession No.:07553), 30Kc19 (GenBank Accession No.:X07554), 30Kc21 (GenBank Accession No.:X07555), 30Kc23 (GenBank Accession No.:X07556), which have common characteristics in amino acid composition and immunological activity, as well as molecular weight. We found that the intracellular expression of 30 K as a representative 30 K protein in mammalian cells inhibits apoptosis in this invention.

Recombinant CHO cell lines producing target proteins are produced by transfecting a target gene into the CHO cell line of the present invention. To mass-produce target proteins industrially using a recombinant CHO cell line, the recombinant CHO cells should be cultured as suspended forms in a serum-free culture media.

When the CHO cell line is cultured as suspended form in a serum-free culture media, the amount of produced target protein tends to decrease due to the apoptosis.

The expression of 30 K resulted in lower intracellular activity for caspase 3. However, the results of in vitro assay of caspase 3 show that the 30 K protein does not inhibit caspase 3 activity. This indicates that the 30 K protein inhibits the apoptosis by working in a further upstream event than caspase 3 activation.

The inhibition of apoptosis is expected to increase in productivity of target proteins by extending longevity of the transfected CHO cell line and to maintain the molecular integrity of unstable target proteins in a medium by decreasing cell lysis.

The present inventors deposited the CHO cell line transfected with 30 K gene at the gene bank of Korea Cell Line Research Foundation (KCLRF-BP-00103). The 30 K gene used in this invention is the DNA of SEQ. ID. No.1.

Another object of the present invention can be achieved by providing the Chinese hamster ovary cell line transfected with 30 K gene, which further containing a gene encoding hematopoietic growth factor.

The hematopoietic growth factor of the present invention is stimulating factor of hematopoiesis. Hematopoiesis is stimulated by a hematopoietic growth factor. Hematopoiesis is the process of renewal and replacement of the cells and formed elements of blood. Blood cells are constantly formed through a process called hematopoiesis.

There are GCSF (Granulocyte colony-stimulating factor), MCSF (Macrophage colony-stimulating factor), EPO (Erythropoietin) and IL (Interleukin), etc. in hematopoietic growth factor.

Erythropoiesis is a subset of this larger scheme and includes only these events that lead from the appearance of the committed erythroid progenitor cell through the formation of mature red blood cells.

The production of red blood cells is stimulated by hormone called erythropoietin which is secreted by the kidneys. The secretion of erythropoietin by the kidneys is stimulated whenever the delivery of oxygen to the kidneys and other organs is lower than normal.

Under these conditions which can occur, for example, when a person lives at high attitude the increased production of red blood cells allows the blood to carry a higher concentration of oxygen to the tissues.

Plasma erythropoietin is a sialoprotein consisting of 165 amino acids. This glycoprotein contains over 40% carbohydrate, consisting of sialic acid and a number of sugars. The sialic acid residues are necessary for biological activity in vivo as in the asialo form it is cleared too rapidly by the liver.

Hereinafter, the present invention will be described in greater detail with reference to the following examples. The examples are given for illustration of the invention and not intended to be limiting the present invention.

### EXAMPLE 1

Cell Line and Culture Condition

The recombinant Chinese Hamster ovary (CHO) cell lines producing human erythropoietin (EPO) and 30 K protein originating from silkworm hemolymph were grown in DMEM/F-12 (1:1) (JRH Bioscience) supplemented with 10% fetal bovine serum (FBS, Gibco), L-glutamine, 15 mM HEPES buffer, and penicillin/streptomycin (Gibco). Cells were Captained at 37° C. in a humidified air atmosphere with 5% CO₂. To produce recombinant human EPO, the growth medium with 10% FBS was replaced with serum-fee medium, EXCELL 301 (JRH Bioscience).

### EXAMPLE 2

Establishment of Stable Recombinant CHO Cell Line Producing 30Kc6

A cDNA clone containing 30Kc6 was kindly provided by S. Izumi (Department of Biology, Tokyo Metropolitan University). He indicated that it was constructed as follows (personal communication): A DNA fragment for one component of 30 K proteins (30Kc6 GenBank accession number, X07552) was amplified by RT-PCR from fifth larval fat body RNA with synthetic oligonucleotide primers specifically used for 30Kc6.

The resulting DNA fragments were digested by EcoRI and inserted into the EcoRI site of pBluescript KS+. We cloned the entire open reading frame of the 30Kc6 into the mammalian expression vector pcDNA3 (Invitrogen).

The pcDNA3/30Kc6 or pcDNA3, the vector alone as a control, was transfected to CHO cells by the LipofectAMINE Reagent (Gibco) according to the manufacturer's instructions. For the establishment of stable cell lines expressing 30 K protein, CHO cells were transfected with the indicated plasmids and moved 48 h later into a selection medium containing 500 μg/mL G418 (Gibco). Selection media were changed every 2-3 days so as to form the colony.

After 3 weeks, clonal selection by picking the colonies was performed for single cell clones. Transfection efficiency was analyzed using a pEGFP expression vector. The cell lines were cotransfected with pEGFP expression vector and test plasmids, and successfully transfected cells were detected by green fluorescent protein (GFP) florescence.

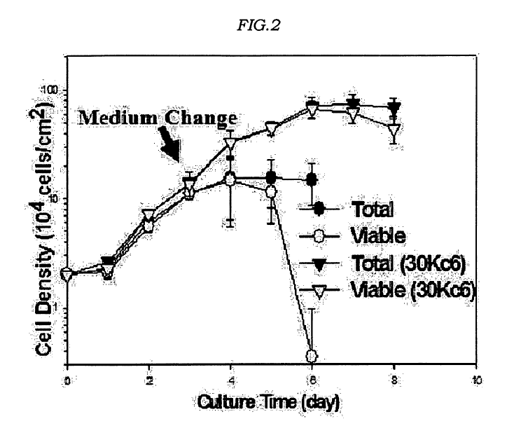

FIG. 2 represents the effect of 30 K protein expression on the cell growth in serum-free medium.

### EXAMPLE 3

RT-PCR

The total cellular RNA was extracted by RNA isolation kit PURESCRIPT (Gentra systems) according to the manufacturer's instructions. RNA concentration was measured spectrophotometrically. Using a sequence specific primer (30Kc6 reverse primer: 5-TCG TTT TCA GCT TCA GCT TTA-3), cDNA was synthesized from 3 μg of total RNA.

PCR was performed for 36 cycles by the following program for each cycle: denaturation at 95° C. for 1 min. annealing at 60° C. for 30 s, and extension at 72° C. for 1 min using a 30Kc6 forward primer (5-ACA GTG TTG TGA CTG cTT TCA-3) and reverse primer (5-TCG TTT TCA GCT TCA GCT TTA-3). The PCR product was analyzed on 1% agarose gel by electrophoresis.

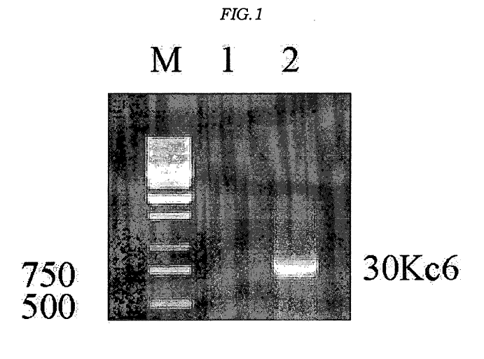

FIG. 1 is the photograph of RT-PCR analysis of 30 K mRNA in the stable CHO cell times transfected with 30 K expression construct. The expected size for the PCR product is 890 bp for the 30 K protein. Lane M, 1 kb molecular weight ladder; lane 1, transfected with pcDNA3; lane 2, transfected with pcDNA3/30Kc6.

### EXAMPLE 4

Establishment of Stable Recombinant CHO Cell Line Producing both 30Kc6 and Human Erythropoietin (EPO) Protein

Human EPO cDNA was amplified by PCR with synthetic oligonucleotide primers and then inserted into Nhe I and Apa I sites of the mammalian expression vector pcDNA3.1/Zeo (+) (Invitrogen). The EPO gene in this plasmid is expressed under the control of human cytomegalovirus immediate-early promoter, and this plasmid confers resistance to Zeocin on the host cells. The pcDNA3.1/Zeo (+)/EPO was transfected to CHO-30Kc6 cell lines expressing silkworm hemolymph 30Kc6 protein by the LipofectAMINE 2000 reagent (Invitrogen) according to the manufacturer's instructions.

For the establishment of stable cell lines expressing both 30 K and EPO, CHO-30Kc6 cells were transfected with the indicated plasmids and moved 48 h later into the selection medium containing 500 μg/ml Zeocin (Invitrogen). Selection media were changed every 2-3 days so as to form the colony. After 3 weeks, clonal selection by picking the colonies was performed for single cell clones.

The clone with highest EPO productivity was selected by the quantitative assay of secreted EPO concentrations in the supernatant. Quantikine IVD EPO ELISA (Enzyme linked immunosorbent assay) (R&D Systems) was used, and it is based on the double-antibody sandwich method.

### EXAMPLE 5

EPO Assay

For the quantitative assay of secreted EPO concentrations in the supernatant, Quantikine IVD EPO ELISA (Enzyme linked immunosorbent assay) (R&D Systems Inc., Minneapolis, Minn.) was used, and it is based on the double-antibody sandwich method Each sample containing EPO secreted from recombinant CHO cells was incubated in microplate wells precoated with monoclonal (murine) antibody specific to EPO. EPO binds to the immobilized antibody on the plate, and an anti-EPO polyclonal (rabbit) antibody-HRP (horse radish peroxidase) conjugate binds to this immobilized EPO.

A chromogen was added into the wells and was oxidized by the enzyme reaction to form a blue colored complex. The reaction was stopped by the addition of acid, which turned the blue to yellow. The amount of color generated is directly proportional to the amount of EPO in the supernatant of production culture. The optical density (O.D.) of each well was determined using a microplate reader (Versamax, Molecular Devices, Sunnyvale, Calif.) set to 450 nm. The EPO concentration was determined by comparing the optical density of the sample to the standard curve. The standards used in this assay were recombinant human EPO calibrated against the Second International Reference Preparation (67/343), a urine-derived form of human EPO.

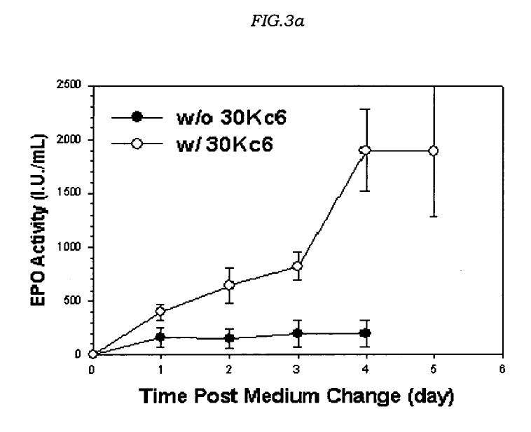

FIG. 3a and FIG. 3b represent the effect of 30 K protein expression on the EPO production in serum-free medium.

### EXAMPLE 6

EPO Glycosylation Assay Using 2-DE

Culture supernatants were concentrated (20×) by centrifugal filter units (Milipore) prior to analysis by two-dimensional electrophoresis (2-DE). For analytical gels, samples of concentrated culture supernatant containing 5000 I.U./mL of EPO were combined with rehydration solution (8.0M urea, 2% CHAPS. 0.3% DTT, 0.5% CA, a few grains of bromophenol blue) in a total volume of 200 μL 7 cm pH 3-10 immobilized pH gradient (IPG) gel strip (Amersham Parmacia Biotech) was rehydrated with this solution overnight. JEF was conducted using an IPGphor unit at 20° C. and the voltage was increased stepwise from 250 V (0.5 h to 500 V (1 h), to 1000 V (1 h), than gradually increased to 8000 V (1 h finally maintained at 8000 V (5 h).

Prior to SDS-PAGE, the LPG gel strips were washed for 15 min in equilibration solution (6.0M urea, 30% glycerol, 2% SDS, 0.05M Tris-HCl) containing 0.1% DTT. This was followed by a 15 min wash in equilibration solution containing 0.25% iodoacetamide. After eqilibration steps, gel strip was loaded on 12% polyacrylamide gel and sealed with 0.5% molten agarose gel.

Proteins separated by SDS-PAGE were electrophoretically transferred onto PVDF membrane. The membrane was blocked by incubation at 4° C. with 5% skim milk for overnight and then incubated with purified mouse monoclonal anti-human EPO (5 mg/mL) at room temperature for at least 3 h Antibody binding was detected by incubation with 1:30,000 diluted alkaline phosphatase-conjugated goat anti-mouse IgG (Santacruz) for 2 h. Nitriblue tetrazolium (0.3 mg/mL; Sigma) and 5-bromo-4-chloro-3-indolyl phosphate (0.15 mg/ml; Sigma) in 5 mM MgCl2 plus 100 mM Tris Buffer were used as a substrate. The membranes were washed 5 times for 5-10 min with PBS/0.19% Tween20 between each step.

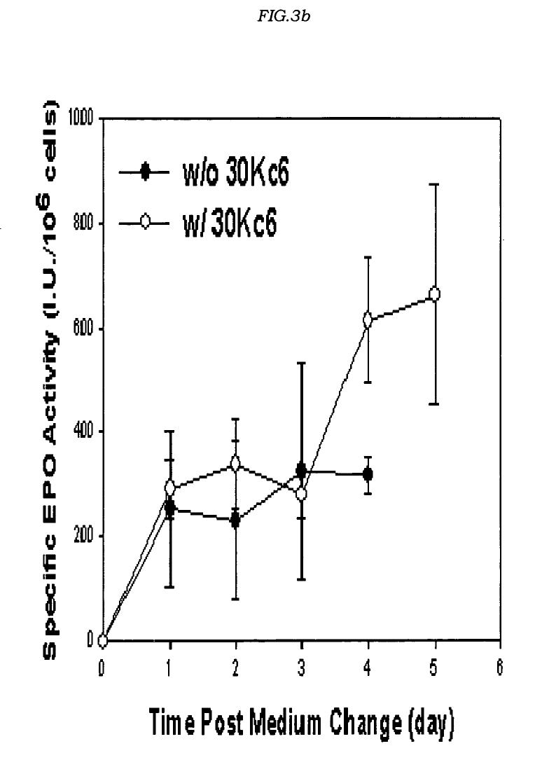

FIG. 4 is a photograph of two-dimensional electrophoresis of EPO samples. Stable expression of 30Kc6 significantly promoted the terminal sialylation of glycans of EPO and reduced the heterogeneity of the glycoforms as shown by a decreased pI range.

### EXAMPLE 7

Sialic Acid Assay Using Lectin

To assay the terminal sialylation of EPO glycans, DIG glycans differentiation kit (Roche) was used. The EPO protein was transferred onto PVDF membrane after SDS-PAGE. Membranes were incubated at 4° C. for overnight in 20 mL blocking solution, and then incubated with 50 μL MAA (Maackia amurensis agglutinin) lectin at room temperature for 1 h. Lectin binding was detected by incubation with anti-digaxigenin-alkaline phosphatase for 1 h. Nitriblue tetrazolium and 5-bromo-4-chloro-3-indolyl phosphate in 5 mM MgCl2 plus 100 mM Tris Buffer were used as a substrate. The membranes were washed 5 times for 5-10 min with TBS buffer (pH 7.5).

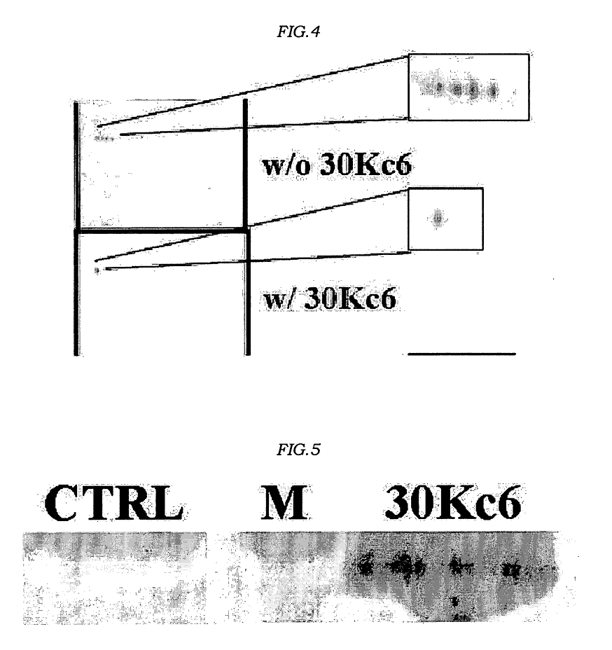

FIG. 5 is a photograph of lectin binding assay of EPO samples. Stable expression of 30Kc6 significantly promoted the terminal sialylation of glycans of EPO as shown by a increased MAA lectin binding.

While the present invention has been described with reference to particular examples thereof, there can be various modifications on the basis of the spirit of the present invention.

What is claimed is:

1. A Chinese hamster ovary cell line (KCLRF-BP-00103) transfected with 30 K gene of SEQ. ID. No.1.

2. The Chinese hamster ovary cell according to claim 1, which further containing a gene encoding hematopoietic growth factor.

3. The Chinese hamster ovary cell line according to claim 2, wherein said hematopoietic growth factor is erythropoietin (EPO) protein.
